(19) United States
(12) Patent Application Publication
(10) Pub. No.: US 2025/0383461 A1
(43) Pub. Date: Dec. 18, 2025

(54) SCANNING METHOD FOR CT DEVICE, PHOTON COUNTING DETECTOR, AND ENERGY SPECTRUM CT SYSTEM

(71) Applicant: WUHAN UNITED IMAGING LIFE SCIENCE INSTRUMENT CO., LTD, Wuhan (CN)

(72) Inventors: Xiaoming Pan, Wuhan (CN); Wenting Xu, Wuhan (CN); Yao Xing, Wuhan (CN)

(21) Appl. No.: 19/315,936

(22) Filed: Sep. 2, 2025

Related U.S. Application Data
(63) Continuation of application No. PCT/CN2024/079827, filed on Mar. 4, 2024.

(30) Foreign Application Priority Data
Mar. 2, 2023 (CN) 202310191821.7

(51) Int. Cl. G01T 1/29

(52) U.S. Cl. CPC G01T 1/2964; G01T 1/2985

(57) ABSTRACT

This disclosure relates to the field of X-ray based medical imaging technology, providing a method, device, and CT scanning imaging system for spectral CT imaging, which may improve the efficiency of reconstructed images. In this disclosure, after obtaining multiple data frames collected by the detector, each data frame is cached in energy segments and stored in memory; Read the data frames required for image reconstruction from memory or disk; Obtain reconstructed images using the read data frames.

### CROSS-REFERENCE TO RELATED APPLICATION

This application is a continuation of the international patent application PCT/CN2024/079827 filed on Mar. 4, 2024 in the United States. The international patent application claims priority to the Chinese patent application with application number 202310191821.7, filed on Mar. 2, 2023 at the China Patent Office. The content of the above application is hereby fully incorporated as a reference.

### TECHNICAL FIELD

The present disclosure relates to the field of medical imaging technology based on X-rays, and in particular, to a scanning method for CT device, photon counting detector and energy spectrum CT system.

### BACKGROUND

Spectral computed tomography (CT) has garnered widespread attention and undergone rapid development in recent years. Currently, spectral CT systems based on photon-counting detectors represent one of the primary implementations. To reduce design and integration complexity and to lower overall costs, each pixel in these photon-counting-detector systems is typically equipped with only a limited number of comparators and counters, for example, two comparators and two counters. Consequently, such systems are ill-suited for applications requiring multiple energy thresholds, such as K-edge imaging or multi-material discrimination. In other words, during a single acquisition, a spectral CT system with few comparators and counters per pixel can only obtain energy bin information at as many discrete thresholds as there are comparators and counters. For example, it may only obtain energy bin information above 30 keV (also known as energy range photon information), where keV denotes one thousand electron volts.

In such cases, spectral CT systems often employ a multi-rotation scanning to acquire energy bin information across multiple energy thresholds. For instance, the first rotation acquires energy bin information above 30 keV, and the second rotation acquires energy bin information above 40 keV. Then, image reconstruction is carried out based on the energy bin information corresponding to multiple energy thresholds.

However, this multi-rotation scanning method introduces a temporal interval between acquisitions of different energy bins, resulting in poor time-domain alignment of the energy bin information, which leads to problems such as motion artifacts, affects the quality of image reconstruction, and also brings additional radiation dose.

### SUMMARY

An aspect of the present disclosure may provide a scanning method for a CT device, which comprises a photon counting detector, wherein the method comprises: obtaining configured threshold information, wherein the threshold information comprises multiple sets of energy thresholds; sending, according to the multiple sets of energy thresholds, a trigger signal to the photon counting detector, wherein the trigger signal is configured to trigger the photon counting detector, sequentially, to adjust a threshold voltage of at least one threshold comparator disposed in the photon counting detector to a voltage corresponding to each of the multiple sets of energy thresholds.

In some embodiments, wherein the sending, according to the multiple sets of energy thresholds, a trigger signal to the photon counting detector, comprises: sending the trigger signal, periodically, to the photon counting detector according to the multiple sets of energy thresholds and a scanning protocol of the photon counting detector.

In some embodiments, wherein the sending the trigger signal, periodically, to the photon counting detector according to the multiple sets of energy thresholds and a scanning protocol of the photon counting detector, comprises: according to the determination that the scanning protocol is a stepwise scanning protocol, sending the trigger signal, periodically, to the photon counting detector based on the number of groups of the energy thresholds and the sampling frequency corresponding to the stepwise scanning protocol.

In some embodiments, wherein the sampling frequency comprises at least one of a first sampling frequency and a second sampling frequency; the first sampling frequency is configured to indicate a time interval between each step angle of the photon counting detector; the second sampling frequency is configured to indicate a data acquisition frequency of the photon counting detector at each of the said step angle.

In some embodiments, wherein the first sampling frequency is a reciprocal of the time intervals between each step angle of the photon counting detector.

In some embodiments, wherein the sending the trigger signal, periodically, to the photon counting detector according to the multiple sets of energy thresholds and a scanning protocol of the photon counting detector, comprises: according to the determination that the scanning protocol is a continuous scanning protocol, sending the trigger signal, periodically, to the photon counting detector according to the number of groups of the energy threshold.

In some embodiments, wherein the sending a trigger signal, periodically, to the photon counting detector according to the number of groups of the energy threshold, comprises: determining a total number of viewing angles for reconstruction of the photon counting detector; sending the trigger signal, periodically, to the photon counting detector according to the total number of viewing angles for reconstruction and the number of groups of the energy threshold.

In some embodiments, wherein the sampling frequency comprises a first sampling frequency, the first sampling frequency is configured to indicate a time interval between each step angle of the photon counting detector; the trigger signal includes a first trigger signal; sending the trigger signal, periodically, to the photon counting detector based on the number of groups of the energy thresholds and the sampling frequency corresponding to the stepwise scanning protocol, comprises: determining, according to the first sampling frequency, whether the photon counting detector has entered the field of view; upon determining that the photon counting detector has entered the said field of view, sending the first trigger signal directly to the photon counting detector, wherein the first trigger signal is configured to trigger the photon counting detector within an acquisition cycle of one field of view, sequentially, to adjust a threshold voltage of at least one threshold comparator disposed in the photon counting detector to a voltage corresponding to each of the multiple sets of energy thresholds.

In some embodiments, wherein the sampling frequency comprises a second sampling frequency, the second sampling frequency is configured to indicate a data acquisition frequency of the photon counting detector at each of the said step angle; said to adjust a threshold voltage of at least one threshold comparator disposed in the photon counting detector to a voltage corresponding to each of the multiple sets of energy thresholds, comprises: to adjust a threshold voltage of at least one threshold comparator disposed in the photon counting detector to a voltage corresponding to each of the multiple sets of energy thresholds based on the second sampling frequency.

In some embodiments, wherein the trigger signal carries the threshold information.

In some embodiments, wherein the sampling frequency comprises at least one of a first sampling frequency and a second sampling frequency; wherein, the first sampling frequency characterizes a rotation angle per sampling of the CT device; the second sampling frequency is configured to indicate a data acquisition frequency of the photon counting detector at each of the said step angle.

In some embodiments, wherein the sending the trigger signal, periodically, to the photon counting detector, comprises sending the trigger signal to the photon counting detector for each preset angle of rotation of the CT device, wherein, a step the preset angle is determined from a total rotation angle, a total number of viewing angles, and the number of groups of the energy threshold for each viewing angle.

Another aspect of the present disclosure may provide a scanning method for a CT device, wherein the CT device comprises a photon counting detector, wherein the method comprises: determining a threshold information, wherein the threshold information comprises multiple sets of energy thresholds; adjusting, sequentially, according to the threshold information, a threshold voltage of a threshold comparator disposed in the photon counting detector to a voltage corresponding to each of the multiple sets of energy thresholds; obtaining a photon information within the energy range corresponding to each of the multiple sets of energy thresholds.

Another aspect of the present disclosure may provide a CT scanning imaging system, includes: a detector configured to collect data frames after scanning; a memory cache unit configured to cache the data frames in an energy-segmented manner; a storage unit configured to store, in an energy-segmented manner, the data frames cached in the memory cache unit; and a processor configured to perform image reconstruction using data frames read from the memory cache unit or the storage unit.

In some embodiments, wherein the adjusting, sequentially, according to the threshold information, a threshold voltage of a threshold comparator disposed in the photon counting detector to a voltage corresponding to each of the multiple sets of energy thresholds, comprises: periodically, adjusting the threshold voltages of the threshold comparator disposed in the photon counting detector, sequentially, to the voltages corresponding to each of the multiple sets of energy thresholds, according to the multiple sets of energy thresholds and a scanning protocol of the photon counting detector.

In some embodiments, wherein, adjusting, sequentially, according to the threshold information, a threshold voltages of a threshold comparator disposed in the photon counting detector to a voltages corresponding to each of the multiple sets of energy thresholds, comprises: converting, according to the multiple sets of energy thresholds, and the mapping relationship between the multiple sets of energy thresholds and the threshold voltages of the threshold comparator, the threshold voltages of the threshold comparator to a voltages of the threshold comparator corresponding to each of the multiple sets of energy thresholds.

Another aspect of the present disclosure may provide a photon counting detector, comprising: a threshold comparator; a determination module, is configured to determine a threshold information, wherein the threshold information includes multiple sets of energy thresholds; an adjustment module, connected to the determination module and the threshold comparator respectively, is configured to adjust a threshold voltage of the threshold comparator disposed in the photon counting detector to a voltage corresponding to each group of the energy thresholds in sequence according to the threshold information; an acquisition module, connected to the threshold comparator, is configured to obtain the photon information of the energy range corresponding to each group of the energy thresholds.

In some embodiments, wherein the adjustment module is also configured to adjust, periodically, the threshold voltages of the threshold comparator disposed in the photon counting detector, sequentially, to the voltages corresponding to each of the multiple sets of energy thresholds, according to the multiple sets of energy thresholds and a scanning protocol of the photon counting detector.

Another aspect of the present disclosure may provide An energy spectrum CT system, comprising: a photon counting detector described above; and/or a computer device described above.

Another aspect of the present disclosure may provide a computing device includes a processor and a memory storing computer program code, wherein the computer program code, when executed by the processor, causes the processor to perform the method described above.

Another aspect of the present disclosure may provide a non-transitory computer-readable storage medium storing computer program code, wherein the computer program code, when executed by a processor, causes the processor to perform the method described above.

Another aspect of the present disclosure may provide a computer program product storing computer program code, wherein the computer program code, when executed by a processor, causes the processor to perform the method described above.

The details of various embodiments of the present disclosure will be explained in the accompanying drawings and description below. Based on the description, drawings, and claims, those skilled in the art will easily understand other features, problems solved, and beneficial effects of the present disclosure.

### BRIEF DESCRIPTION OF THE DRAWINGS

FIG. 1 is a schematic diagram of the disclosure environment of a scanning method for CT device according to an embodiment;

FIG. 2 is a flow diagram of a scanning method for a CT device according to an embodiment;

FIG. 3 is a schematic diagram of the structure of a photon counting detector at a single pixel according to an embodiment.

FIG. 4 is a workflow diagram of a photon counting detector under the stepwise scanning protocol according to an embodiment.

FIG. 5 is a schematic diagram of a process for sending a trigger signal to a photon counting detector according to an embodiment.

FIG. 6 is a workflow diagram of a photon counting detector under the continuous scan protocol according to an embodiment.

FIG. 7 is a schematic flow diagram of another scanning method for a CT device according to an embodiment.

FIG. 8 is a block diagram illustrating the structure of a computer device according to an embodiment.

FIG. 9 is a block diagram of the structure of a photon counting detector according to an embodiment.

FIG. 10 is a schematic diagram of the internal structure of the computer device according to an embodiment.

FIG. 11 is a schematic diagram of the structure of a spectral CT system according to an embodiment.

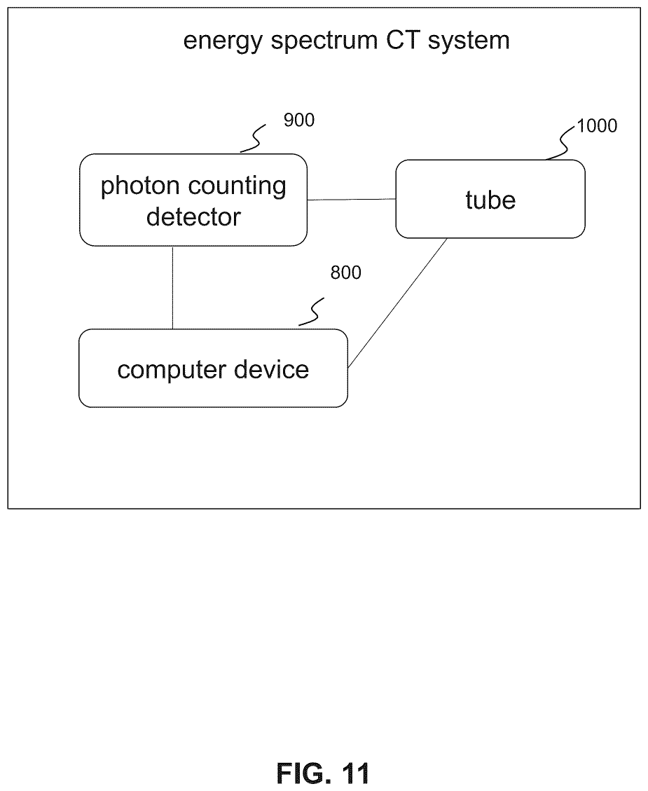

### DETAILED DESCRIPTION

In order that the objects, technical solutions and advantages of the present disclosure will become clearer, the present disclosure will be further described in detail below with reference to the accompanying drawings and embodiments. It should be understood that the specific embodiments described herein are merely for explaining the present disclosure and are not intended to limit the present disclosure.

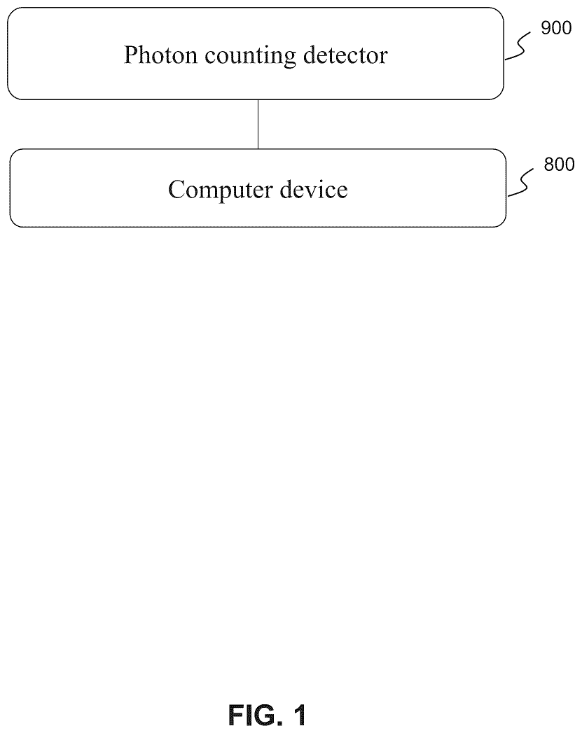

FIG. 1 is a schematic diagram of the disclosure environment of a scanning method for a CT device in an embodiment of the present disclosure. In this case, the photon counting detector 900 communicates with the computer device 800 either wired or wirelessly. The computer device 800 may, but is not limited to, all kinds of personal computers, laptops, smartphones, tablets and portable wearable devices. Portable wearable devices may be smartwatches, smart bands, headsets, etc. Of course, computer Device 800 may also be implemented with a standalone server or a server cluster of multiple servers.

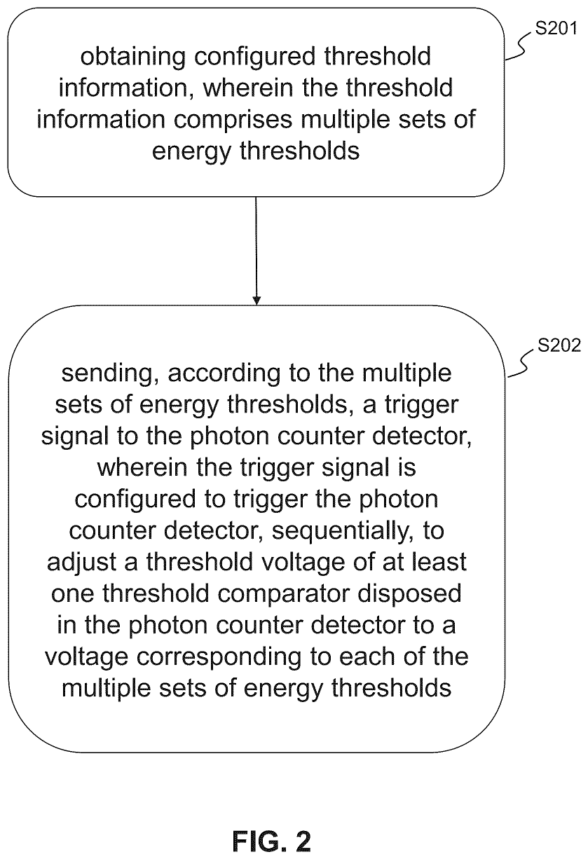

FIG. 2 shows a flowchart of a scanning method for a CT device in an embodiment of the present disclosure, all or part of the steps of which may be applied to the computer device 800 shown in FIG. 1, and all or part of the steps of which may also be applied to the photon counting detector 900. Of course, some of the steps of the method may also be performed by other components or modules of the CT device. In one embodiment, as shown in FIG. 2, the scanning method for the CT device includes steps S201 and S202.

In step S201, obtaining configured threshold information, wherein the threshold information comprises multiple sets of energy thresholds.

The following concepts are introduced first. An energy threshold refers to the starting point of an energy range. For example, an energy threshold of 30 keV indicates an energy range starting at 30 keV. Energy bin information refers to the bin information corresponding to an energy range. The energy bin information corresponding to the energy threshold represents the bin information corresponding to the energy range starting from that energy threshold. For example, the energy bin information corresponding to 30 keV represents the bin information corresponding to the energy range above 30 keV. Energy bin information may also be referred to as energy range photon information. A single scan refers to a single scan of a CT device that includes a photon counting detector, which is related to the user's scanning requirements. A single scan may be a multi-rotation scan, such as a spiral scan, or a 360° scan, or a half-rotation or quarter-rotation scan, etc.

In this embodiment, taking the scanning method of the CT device executed by the computer device as an example, when the photon counting detector needs to obtain the energy bin information corresponding to multiple energy thresholds, the computer device will first obtain the configured threshold information. In some embodiments, the computer device may provide an interactive interface based on which the user configures the threshold information, and the computer device may also receive threshold information sent from other electronic devices, such as a USB flash drive.

The threshold information includes multiple sets of energy thresholds, that is, multiple groups of energy thresholds, which are used to instruct the photon counting detector to obtain the energy bin information corresponding to each of the multiple sets of energy thresholds.

In some embodiments, the number of energy thresholds included in each group of energy thresholds may be related to the photon counting detector, for example, the number of energy thresholds included in each group of energy thresholds is related to the structure of the photon counting detector. Specifically, the maximum amount of energy bin information that a photon counting detector may currently obtain in a single scan is related to the number of single-pixel comparators and counters in the photon counting detector. A set of single-pixel comparators and counters in a photon counting detector may correspond to a set of energy thresholds, and each set of energy thresholds may correspond to a set of energy bin information. In some embodiments, the threshold information includes multiple sets of energy thresholds, and the number of energy thresholds in each set of energy thresholds may be the same as the number of comparators in the photon counting detector, and each comparator in the photon counting detector may correspond to a counter.

For example, if photon counting detector A has two comparators and two counters, then currently photon counting detector A may obtain energy bin information corresponding to up to two energy thresholds in a single scan. If the user wants to obtain energy bin information corresponding to multiple energy thresholds such as 20 keV, 30 keV, 40 keV, 50 keV, 60 keV, and 70 keV in a single test for photon counting detector A, the user may input “20”, “30”, “40”, “50”, “60” and “70” to the computer device via the interactive interface provided by the computer device. The computer device may obtain threshold information including three sets of energy thresholds, each set consisting of two energy thresholds. For example, Group one may include energy thresholds “20 kev” and “30 Kev”, Group 2 may include energy thresholds “40 keV” and “50 keV”, and Group 3 may include energy thresholds “50 keV” and “60 keV”.

It should be noted that the above said is only an exemplary implementation, and the order of each energy threshold in each energy threshold group may be set according to actual requirements. Of course, the user may configure the threshold information by other means, such as by voice input, which is not limited to the embodiments of the present disclosure.

In some embodiments, the computer device may store the threshold information in the form of structured arrays, etc. One form in which the computer device acquires multiple sets of energy thresholds is: The computer device acquires “{Threshold-one, Threshold-two}, {Threshold-three, Threshold-four}, {Threshold-five, Threshold-six}, etc.” As in the example above, the computer device may take 20 as the value of Threshold-one, 30 as the value of Threshold-two, and so on. Here, each parenthesis marks a set of energy thresholds, that is, an energy threshold group, and Threshold-one to Threshold-six respectively represent different energy thresholds, that is, the starting points of different energy ranges.

It should be noted that the number of energy thresholds in each group of energy thresholds may be the same or different. When the number of energy bin information that the user wants to obtain may divide the total number of comparators in the photon counting detector, the number of energy thresholds in each group is the same. Otherwise, the number of energy thresholds in each group may be different. For example, if the user wants to obtain the energy bin information corresponding to five different energy thresholds and the photon counting detector only includes two comparators, the number of energy thresholds in each group of energy thresholds will be different.

In step S202, sending, according to the multiple sets of energy thresholds, a trigger signal to the photon counting detector, wherein the trigger signal is configured to trigger the photon counting detector, sequentially, to adjust a threshold voltage of at least one threshold comparator disposed in the photon counting detector to a voltage corresponding to each of the multiple sets of energy thresholds.

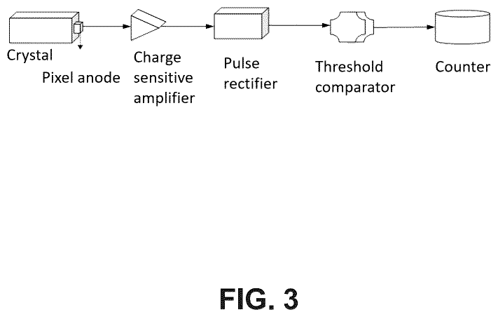

FIG. 3 shows a schematic diagram of the structure of a photon counting detector at a single pixel. As shown in FIG. 3, the photon counting detector at a single pixel comprises a crystal, a charge-sensitive amplifier, a pulse rectifier, a threshold comparator, and a counter.

The photon counting detector shown in FIG. 3 detects photons during scanning, which are converted into large carriers by the crystal and received as current pulses by the anodes of the pixels on the crystal. These current pulses are amplified by the charge-sensitive amplifier and rectified by the pulse rectifier into voltage pulses.

Further, the voltage pulse input to the threshold comparator is compared with the threshold voltage of that threshold comparator, and the counter counts the pulse events higher than that threshold voltage, that is, counts the number of photons that meet the energy threshold requirements corresponding to the threshold voltage. Ultimately, the counter counts the energy bin information corresponding to different energy thresholds and feeds the energy bin information into the acquisition system to obtain the projection data of the CT device for subsequent image reconstruction work. In the related technology, the threshold voltage of the photon counting detector usually remains constant in a single scan. Therefore, energy spectrum CT with a limited number of threshold comparators and counters may only obtain energy bin information corresponding to a fixed energy threshold in a single experiment. For example, a single energy CT may only obtain energy bin information above 30 keV in one scan.

In this embodiment, after obtaining the configured threshold information, the computer device sends trigger signals to the photon counting detector based on multiple sets of energy thresholds in the threshold information, and the photon counting detector adjusts the threshold voltage of its own threshold comparator to the voltages corresponding to each set of energy thresholds in sequence according to the trigger signals, that is, in a single scan of the photon counting detector, it modifies the threshold voltage of the threshold comparator, thereby expanding the energy bin information that the photon counting detector may obtain in a single scan.

In one embodiment, the computer device may send a trigger signal to the photon counting detector based on the configured threshold information and the scanning protocol of the photon counting detector, or the computer device may also send a trigger signal to the photon counting detector at a preset sending frequency. The threshold information may be carried in the trigger signal, and the trigger signal and the threshold information may be sent separately.

In some embodiments, the threshold information also includes the voltage of the threshold comparator corresponding to the energy threshold. The photon counting detector may also store the mapping relationship between the energy threshold of the photon counting detector and the voltage of the threshold comparator. Threshold information includes multiple sets of energy thresholds, and each energy threshold may obtain the corresponding threshold comparator voltage through this mapping relationship. In some embodiments, the mapping relationship between the multiple sets of energy thresholds stored by the photon counting detector and the voltage of the threshold comparator may be utilized to convert the received multiple sets of energy thresholds into the voltage of the corresponding threshold comparator, that is, the threshold voltage. By using different threshold voltages, the photon information of the energy range corresponding to each energy threshold may be obtained to complete the image acquisition of multiple groups of different energy ranges.

For the example of the step S201 mentioned above, if the threshold information obtained by the computer device is “{20,30}, {40,50}, {60,70}”, the computer device sends trigger signal to the photon counting detector A every five milliseconds, then the photon counting detector, during a single scan, receives the trigger signal sent by the computer device every five milliseconds, where five milliseconds is only used for an exemplary description, and the actual period during which the computer device sends the trigger signal depends on the sampling period of the photon counting detector A. Further, the photon counter A adjusts the threshold voltage of the threshold comparator to the voltage corresponding to each group of energy thresholds in sequence every five milliseconds based on a trigger signal and threshold information, from the single scan beginning. The trigger signal may be based on external hardware triggering, internal software triggering, or internal clock triggering, among which the external hardware triggering may be based on level triggering, edge triggering, or pulse triggering.

For example, the photon counting detector A includes two sets of threshold comparators as shown in FIG. 3 at a single pixel, that is, the photon counting detector A includes threshold comparator 1 and threshold comparator 2. When photon counting detector A executes a single scan, it receives a trigger signal and then adjusts the threshold voltages of threshold comparator 1 and threshold comparator 2 to voltages corresponding to 20 keV and 30 keV respectively, to obtain the energy bin information above 20 keV and above 30 keV. Next, Photon counting detector A adjusts the threshold voltages of threshold comparator 1 and threshold comparator 2 to the voltages corresponding to 40 keV and 50 keV respectively to obtain the energy bin information above 40 keV and above 50 keV. Finally, the Photon counting detector A adjusted the threshold voltages of threshold comparator 1 and threshold comparator 2 to 60 keV and 70 keV respectively to obtain energy bin information above 60 keV and above 70 keV. After five milliseconds, the computer device sends the next trigger signal. In this example, five milliseconds contains m sets of time for data acquisition and readout, that is, the trigger period is m times that of data sampling period, the time for the threshold comparator to respond to the trigger signal and adjust the voltage is so short that it may be temporarily disregarded. And so on, each time photon counting detector A receives a trigger signal, it adjusts the threshold voltage of the threshold comparator in sequence to the voltage corresponding to each group of energy thresholds.

In this way, by quickly switching the threshold voltage of the threshold comparator in a single scan, energy bin information at different energy thresholds may be obtained. It is understandable that the photon counting detector A first gets energy bin information for 20 keV and 30 keV, then for 40 keV and 50 keV, and finally for 60 keV and 70 keV. Photon counting detector A may also obtain energy bin information in other orders, as long as it may obtain energy bin information corresponding to each energy threshold in the end.

In some embodiments, in order to improve the quality of subsequent image reconstruction, the threshold voltages of different threshold comparators in the photon counting detector are switched simultaneously during each round of threshold voltage switching. For example, the threshold voltage of threshold comparator 1 is adjusted to the voltage corresponding to 20 keV. It is carried out and completed simultaneously with the threshold voltage adjustment of 30 keV in threshold comparator 2.

In some embodiments, the computer device may perform the threshold comparison function by digitizing the input signal through a digital to analog converter (DAC) in the threshold comparator for comparison with the threshold voltage. The threshold voltage of the threshold comparator is controlled by a digitally controlled voltage divider module (or chip).

The above is illustrated by the example of a computer device directly controlling the threshold voltage of the threshold comparator in a photon counting detector through a trigger signal. In some embodiments, the computer device may also indirectly control the threshold voltage of the threshold comparator in a photon counting detector through a trigger signal. For example, after receiving the trigger signal sent by the computer device, the photon counting detector also needs to determine by its own internal clock when to adjust the threshold voltage of the threshold comparator to the voltages corresponding to each energy threshold in sequence.

The scanning method for CT device provided in the embodiments of the present disclosure, acquires configured threshold information, which includes multiple sets of energy thresholds, and then sends trigger signals to the photon counting detector based on the multiple sets of energy thresholds. The trigger signal is used to trigger the photon counting detector to adjust the threshold voltage of the threshold comparator in the photon counting detector to the voltage corresponding to each energy threshold in sequence. In a single scan of the current photon counting detector, the threshold voltage of the threshold comparator does not change. That is to say, assuming the photon counting detector may obtain energy bin information corresponding to up to 2 energy thresholds in a single scan, if the user wants energy bin information corresponding to 4 energy thresholds, the photon counting detector has to perform two separate single scans. The first scan acquires the energy bin information corresponding to energy threshold 1 and energy threshold 2, and the second scan acquires the energy bin information corresponding to energy threshold 3 and energy threshold 4. Since there is at least one single scan time between the first and second scans. As a result, the time-domain matching of energy bin information corresponding to multiple energy thresholds in current scanning methods is poor.

In the embodiment of the present disclosure, by sending a trigger signal to the photon counting detector and using the trigger signal to trigger the photon counting detector to adjust the threshold voltage of the threshold comparator, the voltages corresponding to each group of energy thresholds may be obtained in a single scan of the photon counting detector, thereby obtaining the energy bin information corresponding to each energy threshold. Therefore, the scanning method for CT device provided in this embodiment may improve the time-domain matching of energy bin information corresponding to multiple energy thresholds, thereby improving the quality of image reconstruction.

In one embodiment, the above step S202, which the sending, according to the multiple sets of energy thresholds, a trigger signal to the photon counting detector, comprises: sending the trigger signal, periodically, to the photon counting detector according to the multiple sets of energy thresholds and a scanning protocol of the photon counting detector.

In this embodiment, in order to better match the working characteristics of the photon counting detector in the process of adjusting the threshold voltage, the computer device periodically sends a trigger signal to the photon counting detector according to the scanning protocol of the photon counting detector and the multiple sets of energy thresholds. The scanning protocol of the photon counting detector may be either a stepwise scanning protocol or a continuous scanning protocol. First determine the type of scanning protocol for the photon counting detector. For example, if the scanning protocol of the photon counting detector is a stepwise scanning protocol, the computer device may send the trigger signal based on the sampling frequency and/or sampling period corresponding to the step-scan protocol.

In one embodiment, the sending the trigger signal, periodically, to the photon counting detector according to the multiple sets of energy thresholds and a scanning protocol of the photon counting detector above-mentioned may be achieved in the following way: if the scanning protocol is a stepwise scanning protocol, a trigger signal is sent periodically to the photon counting detector based on the number of groups of energy thresholds and the sampling frequency and/or sampling period corresponding to the stepwise scanning protocol.

In this embodiment, the stepwise scanning protocol refers to the protocol in which the photon counting detector completes a single scan at a certain step angle. For example, a photon counting detector needs to scan 360° in a single scan. If the photon counting detector scans according to the stepwise scanning protocol with a step angle of 5° and scans for one second at each step angle, then the photon counting detector will complete a single scan of the entire 360° by stopping for one second for every rotation of 5°.

In one embodiment, the sampling frequency includes the first sampling frequency and/or the second sampling frequency. In which, the first sampling frequency is configured to indicate a time interval between each step angle of the photon counting detector. For example, the first sampling frequency may be the reciprocal of the time interval between each step angle. The time interval between each step angle may be the sum of the sampling time the photon counting detector stays at each step angle for sampling and the rotation time it takes to rotate each step Angle. For example, if the photon detector samples at a step angle of 5°, the pause time at each step angle is the sampling time of one second, and the rotation time from that angle of 5° to the next sampling angle after each angle of data is collected is one second. Then the time interval for each step angle is two seconds. The second sampling frequency is configured to indicate a data acquisition frequency of the photon counting detector at each of the said step angle, for example, the data acquisition frequency may be the reciprocal of the time period corresponding to the acquisition of each frame of the image.

In one embodiment, the first sampling frequency is used to characterize that for each rotation angle of the CT device, a trigger signal is sent to the photon counting detector causing the photon counting detector to perform one sampling, with the rotation angle determined by the number of views in a single circle. For example, for every 5° rotation of the CT device, a certain period of time, that is, the sampling time, is used for sampling. After collecting data for rotation 5°, the CT device continues to rotate and stops for a certain period of time for sampling at every rotation 5° of the device. In this way, the first sampling frequency is to sample once for every rotation 5° of the CT device.

Further, the computer device may periodically send trigger signals to the photon counting detector based on the number of groups of energy thresholds and the first and/or second sampling frequencies corresponding to the stepwise scanning protocol.

In some embodiments, the computer device may periodically send trigger signals to the photon counting detector based on the number of groups of the first sampling frequency and energy threshold corresponding to the stepwise scanning protocol. According to the first sampling frequency, the computer device confirms whether the photon counting detector has entered each view. If it has entered each view, it may directly send the trigger signal to the photon counting detector. The trigger signal may be the first trigger signal used to trigger a photon counting detector to complete data acquisition at one view within one view acquisition cycle. Then resume data acquisition for the next view. Each time a view is entered, the computer device sends a first trigger signal to the photon counting detector. At each view, the voltages corresponding to each group of energy thresholds may be adjusted directly according to the clock control of the photon counting detector. For example, with a step angle of 5°, the first trigger signal is sent for each rotation 5° of the CT device. The 1st first trigger signal may be sent at the moment when the rotation angle 0° (0° is the initial Angle). In the view 0°, the voltages corresponding to the energy thresholds of each group are adjusted according to the clock control of the photon counting detector. After the data collection in this view is completed, the CT device rotates 5° to enter the next view and sends the 2nd first trigger signal. At view 5°, the voltage corresponding to each energy threshold is adjusted according to the clock control of the photon counting detector. After completing the data acquisition at this view, so on, to complete the data acquisition at all views.

In some embodiments, the computer device may periodically send trigger signals to the photon counting detector according to the number of groups of the second sampling frequency and energy threshold corresponding to the stepwise scanning protocol. In this embodiment, the CT system may automatically control the photon counting detector to enter each view according to the user's settings. In each view, according to the data acquisition frequency, a trigger signal may be sent to the photon counting detector after each acquisition of m frames of image data. Wherein the m may be a natural number greater than or equal to one, which may be set automatically by the system or manually. Based on the trigger signal, adjust the threshold voltage of the threshold comparator in the photon counting detector in sequence to the voltage corresponding to each group of energy thresholds. The trigger signal may be a second trigger signal used to adjust the threshold voltage of the threshold comparator in the photon counting detector to the voltages corresponding to each energy threshold in sequence. In one acquisition cycle, the number of times the second trigger signal may be matched with the number of groups of energy thresholds. It is understandable that the matching may be the same as the number of times the second trigger signal is used to match the number of groups of the energy threshold.

For example, the computer device may send out the 1st second trigger signal as soon as the photon counting detector enters a view acquisition, adjusting the threshold voltage of the threshold comparator in the photon counting detector to the voltage corresponding to the first set of energy thresholds, at which m frames of image data are collected. Then, a second 2nd trigger signal is sent to adjust the threshold voltage of the threshold comparator in the photon counting detector to the voltage corresponding to the second set of energy thresholds, and so on, the number of second trigger signals may be the same as the number of sets of energy thresholds until the data acquisition at that view is completed, completing one acquisition cycle. In this implementation, the 1st second trigger signal in each view may be controlled according to the rotation angle.

In some embodiments, the computer device may periodically send trigger signals to the photon counting detector according to the first sampling frequency, the second sampling frequency, and the number of groups of energy thresholds corresponding to the stepwise scanning protocol. In this embodiment, the threshold voltages of the threshold comparators in the photon counting detector may be adjusted in sequence to the voltages corresponding to each energy threshold based on the first and second trigger signals.

For example, a first trigger signal may be sent at the beginning of a view acquisition to adjust the threshold voltage of the threshold comparator in the photon counting detector to the voltage corresponding to the first set of energy thresholds, at which m frames of image data may be collected. Then, based on the first second trigger signal, adjust the threshold voltage of the threshold comparator in the photon counting detector to the voltage corresponding to the second set of energy thresholds, with the number of second trigger signals being the number of sets of energy thresholds minus one. Until the data acquisition at this view is completed, one acquisition cycle is finished. Move on to the next acquisition view and then trigger the second first trigger signal to enter the acquisition cycle of the next view. Repeat this cycle until all data acquisition at step angles is completed.

It should be noted that the first sampling frequency and/or the second sampling frequency may be a user-defined frequency received by the computer device, or a frequency determined by the photon counting detector itself and sent to the computer device.

Since the trigger signal is sent periodically to the photon counting detector according to the stepwise scanning protocol corresponding to the first sampling frequency and/or the second sampling frequency, the timing of sending the trigger signal in this implementation is relatively accurate, so that the threshold voltages of the threshold comparator in the photon counting detector may be precisely adjusted in sequence to the voltages corresponding to each energy threshold.

In some embodiments, if the scanning protocol is a stepwise scanning protocol, the trigger signal may also be sent periodically to the photon counting detector according to the number of groups of energy thresholds and the sampling period corresponding to the stepwise scanning protocol.

In some embodiments, the sampling period includes the time interval between each step angle of the photon counting detector. For example, the time interval between each step angle may be the sum of the sampling time the photon counting detector stays at each step angle for sampling and the rotation time for rotating each step angle.

In some embodiments, the sampling period includes the first sampling period and/or the second sampling period. Here, the first sampling period is the time interval between each step angle of the photon counting detector. The second sampling period is the data acquisition period of the photon counting detector at each of the said step angles. It is understandable that the principle of sending the trigger signal according to the sampling period is similar to the principle of sending the trigger signal according to the sampling frequency.

Take the example of a computer device periodically sending a trigger signal to a photon counting detector based on the number of groups of the first sampling period and energy threshold corresponding to the stepwise scanning protocol. After the photon counting detector starts working, the computer device determines whether the photon counting detector is at view one based on the first sampling period. When the photon counting detector enters view one, it sends the first trigger signal one to the photon counting detector, which is controlled by the photon counting detector at view one according to its own clock. Adjusting the threshold voltage of the threshold comparator in sequence to obtain the information of each energy bin corresponding to multiple sets of energy thresholds at view one. In the same way, the computer device determines whether the photon counting detector has entered each view based on the first sampling period. If it has entered each view, it sends the first trigger signal to the photon counting detector, and the photon counting detector, upon receiving the first trigger signal, directly adjusts the voltage corresponding to each energy threshold based on its own clock control. In order to be able to obtain the energy bin information corresponding to multiple sets of energy thresholds in each view.

Taking the periodic sending of trigger signals by the computer equipment to the photon counting detector based on the number of groups corresponding to the second sampling period and energy threshold of the step scanning protocol as an example, the CT system automatically controls the photon counting detector to enter view one according to the user's settings. After the photon counting detector enters view one, the computer device sends the second trigger signal one to the photon counting detector. The photon counting detector adjusts the threshold voltage to the voltage corresponding to the first group of energy thresholds, thus completing one acquisition cycle, where the acquisition cycle is the second sampling period, and the information of each energy bin corresponding to the first group of energy thresholds is obtained. Then the computer device sends a second trigger signal two to the photon counting detector, which adjusts the threshold voltage to the voltage corresponding to the second energy threshold, thus completing the second acquisition cycle and obtaining the information of each energy bin corresponding to the second energy threshold. And so on, until data acquisition at all step angles is completed and multiple sets of energy bin information corresponding to energy thresholds are obtained at each view.

In some embodiments, the computer device may also periodically send trigger signals to the photon counting detector based on the first sampling period, the second sampling period, and the number of groups of energy thresholds corresponding to the stepwise scanning protocol, indicating that the photon counting detector has completed the regulation of the voltage corresponding to a set of energy thresholds after entering each view and during the acquisition period within each view.

It should be noted that the trigger signal may be sent by the computer device and/or the photon counting detector. In some embodiments, the trigger signal may be a signal periodically sent by the computer device to the photon counting detector based on multiple sets of energy thresholds. In some embodiments, the trigger signal may also be a periodic signal determined by the photon counting detector based on multiple sets of energy thresholds and the internal clock. In some embodiments, the trigger signal may also be the signal periodically sent by the computer device to the photon counting detector based on multiple sets of energy thresholds and the periodic signal determined by the photon counting detector based on multiple sets of energy thresholds and the internal clock. Of course, in some embodiments, the trigger signal may be triggered by other control components of the CT device.

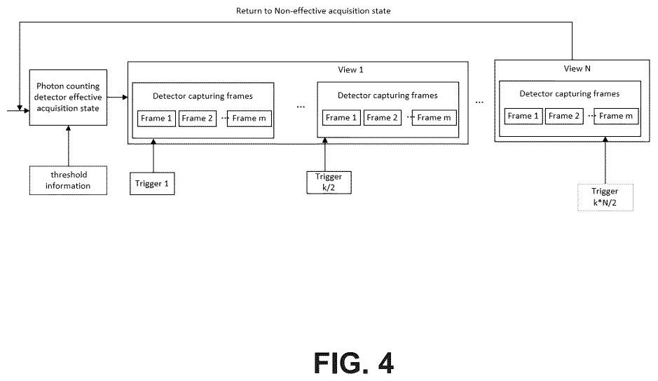

FIG. 4 shows a workflow diagram of a photon counting detector in one embodiment of the present disclosure under the stepwise scanning protocol. As shown in FIG. 4, the photon counting detector may change from the non-effective acquisition state to the effective acquisition state after receiving the threshold information sent by the computer device. Here, the effective acquisition state refers to the state where the photon counting detector is collecting valid data, and the non-effective acquisition state refers to the state where the photon counting detector is collecting non-valid data or is not in the acquisition state. Valid data includes data that may be used for image reconstruction.

Further, the computer device may periodically send a trigger signal to the photon counting detector according to the sampling frequency corresponding to the stepwise scanning protocol.

In FIG. 4, N is the number of views generated by the photon counting detector under the stepwise scanning protocol, N is a preset value, which is an integer greater than 0. For example, if the preset number of views N=72, then the photon counting detector has a step angle of 5° in one 360° scan. k is the total number of energy thresholds in the threshold information, an integer greater than or equal to 1. m is the number of data frames collected for each energy threshold at each step angle, and m may be a preset integer greater than or equal to 1.

Further, for photon counting detector A, the threshold information includes threshold-1, threshold-2, threshold-3, threshold-4, threshold-5, and threshold-6, that is, k=6. Single scanning is 360°, and the number of preset views N=72. Each group of energy thresholds contains two thresholds, that is, the number of groups of energy thresholds is three. When the rotation angle of the CT device is 0° (that is, view one), the computer device sends a trigger signal one to the photon counting detector A. At this time, the photon counting detector A first adjusts the threshold voltages of threshold comparator one and threshold comparator two respectively according to the voltages corresponding to threshold one and threshold two. Then m frames of images are captured at threshold voltage one and threshold voltage two respectively. Then the computer device sends trigger signal two to the photon counting detector A, which adjusts the threshold voltages of threshold comparator one and threshold comparator two respectively according to the voltages corresponding to threshold three and threshold four, repeating the previous set of acquisition timing. Finally, the computer device sends trigger signal three to the photon counting detector A, which adjusts the threshold voltages of threshold comparator one and threshold comparator two respectively according to the voltages corresponding to threshold five and threshold six. Repeat the previous set of acquisition timing to capture m frames at threshold voltage five and threshold voltage six respectively. The above trigger signal one, trigger signal two, and trigger signal three are the second trigger signals.

In this way, the photon counting detector A acquires the energy bin information corresponding to threshold-1, threshold-2, threshold-3, threshold-4, threshold-5, and threshold-6, at view one respectively. Then, when the photon counting detector A rotates 5° (that is, view 2), the computer device sends trigger signal four-trigger signal six to the photon counting detector so that the photon counting detector A acquires the energy bin information corresponding to view two at Threshold-1-Threshold-6 respectively. In the same way, the photon counting detector A may obtain N*k*m frames of images at N perspectives and return the non-effective acquisition state after obtaining N*k*m frames of images at N perspectives.

In this example, the scanning protocol is a stepwise scanning protocol, and trigger signals are periodically sent to the photon counting detector based on the number of groups of energy thresholds and the sampling frequency and/or sampling period corresponding to the stepwise scanning protocol, further improving the time-domain matching of energy bin information. Meanwhile, for the photon counting detector under the step scan protocol, it saves a amount of step rotation time compared to the multi-rotations sequence scan, which is of great significance for some time-limited in vivo drug diagnoses.

In one embodiment, the aforementioned sending the trigger signal, periodically, to the photon counting detector according to the multiple sets of energy thresholds and a scanning protocol of the photon counting detector may also be achieved by sending a trigger signal periodically to the photon counting detector based on the number of sets of energy thresholds, if the scanning protocol is a continuous scanning protocol.

In this embodiment, the continuous scan protocol includes the axial scan protocol and the helical scan protocol. A continuous scan protocol refers to a protocol in which a photon counting detector scans without stopping while rotating in order to continuously complete a single scan. In other words, under the continuous scan protocol, the photon counting detector does not stop rotating while scanning a 360° circle. Therefore, if the scanning protocol is a continuous scan protocol, the computer device will periodically send a trigger signal to the photon counting detector based on the number of groups of energy thresholds. The period for sending the trigger signal to the photon counting detector may be determined based on the rotational speed of the photon counting detector, according to the rotational time of the photon counting detector. In one embodiment, the period for sending the trigger signal to the photon counting detector may also be determined based on the angle of rotation of the CT device. For each preset angle the CT device rotates, a trigger signal is sent to the photon counting detector, and based on the trigger signal, the photon counting detector changes the threshold voltage and acquires the image. Under the continuous scan protocol, the photon counting detector captures a set of threshold images at each angle step, and multiple sets of threshold images are achieved by capturing them at multiple adjacent angles. The angle intervals and time intervals for capturing multiple sets of threshold images are very short, and the captured multiple sets of threshold images may be myopically regarded as data at the same viewing angle. The preset angle step is determined by the total rotation angle, the total number of viewing angles, and the number of groups of energy thresholds for each viewing angle. In this way, when the rotation speed of the photon counting detector is uneven, a trigger signal is sent periodically to the photon counting detector with the preset angle as the standard to ensure the quality of image acquisition.

For example, if the total rotation angle is 360°, the total number of viewing angles is 90, and the number of groups of energy thresholds for each viewing angle is four, the preset angle step is 1°, that is, for every rotation 1° of the CT device, the computer equipment sends a trigger signal to the photon counting detector. The CT device may obtain four sets of different threshold acquisition data at [0°,1°,) [1°,2°), [2°,3°), [3°,4°), approximately as multi-energy threshold data for the first view, and so on for subsequent angles. In practical use, the angle intervals between these four sets of data may be smaller.

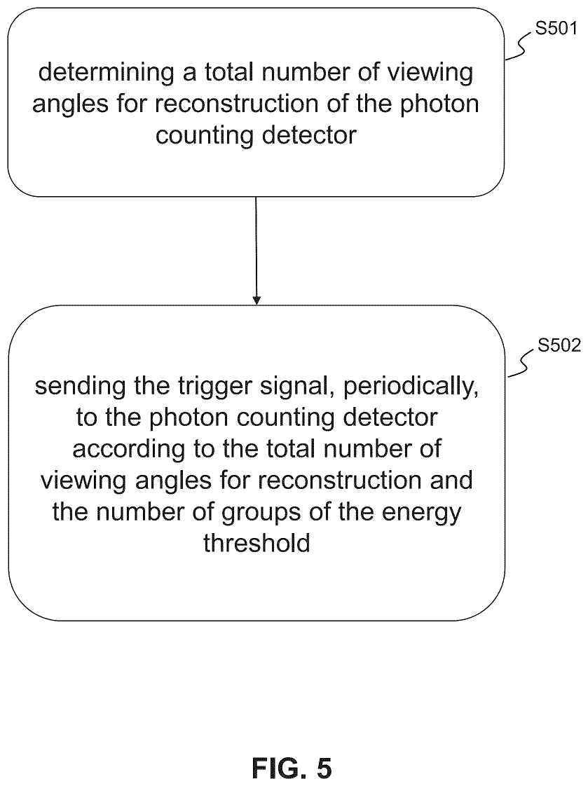

FIG. 5 shows a schematic diagram of the process of sending a trigger signal to a photon counting detector in an embodiment of the present disclosure. With reference to FIG. 5, this embodiment relates to an implementation of how to periodically send a trigger signal to a photon counting detector. Based on the above embodiments, the above sending a trigger signal periodically to the photon counting detector based on the number of sets of energy thresholds comprises steps S501 and S502.

Step S501, determining a total number of viewing angles for reconstruction of the photon counting detector.

In this embodiment, the computer equipment needs to determine the total number of viewing angles for reconstruction of the photon counting detector. The total number of views angles for reconstruction of the photon counting detector is the total number of views for image reconstruction of the photon counting detector under the continuous scan protocol. For example, if the photon counting detector scans a circle of 360° in a single scan under the continuous scan protocol and the total number of views for reconstruction is 360, then each view contains 1° of scanning data.

Step S502, sending the trigger signal, periodically, to the photon counting detector according to the total number of viewing angles for reconstruction and the number of groups of the energy threshold.

In some embodiments, the trigger period of the trigger signal may be expressed as:

$${T}_{v} = \frac{{T}_{k}}{q} .$$

Wherein, Tₖ represents the rotation period of the computer device for each reconstruction view corresponding to the scanning protocol, q represents the number of groups of energy thresholds. In one of these scenarios, the value of Tₖ determined by the CT device. Tₖ may be determined based on the total rotation angle, the total number of views, and the rotational speed of the CT device, that is, based on the rotational speed of the CT device and the rotation angle of each reconstruction view.

For example, in a continuous scanning protocol, the total angle of the scanning protocol is 360°, the total number of views for reconstruction is 90, and the rotational speed of the CT device is 1°/ms, then for each rotation period of the view for reconstruction, the number of energy threshold groups is 4, the Tₖ=4 ms, then the CT device sends a trigger signal to the photon counting detector every 1 ms, and controls the detector to modify the threshold and capture the image once.

In continuous scanning protocol, the rotational speed of the CT device may be regarded as quasi-uniform, and when precision requirements are not high, it may be approximated as uniform. When higher precision is required, small angular errors also need to be taken into account, and the trigger period may be determined based on the angular step. In some embodiments, the trigger period may be determined based on the angle step of the scanning protocol, and the trigger signal is sent to the photon counting detector according to the trigger period. For example, the total rotation angle of the scanning protocol is 360°, the total number of viewpoints for reconstruction is 360, the number of energy threshold groups is 3, the total number of angle steps is 3*360=1080, the angle step is ⅓ degree, the CT device sends a trigger signal to the photon counting detector every ⅓ degree to control the detector to modify the threshold and capture the image once. Of course, this implementation may also be applied to scanning protocols that rotate at a constant speed.

Also, since the continuous scanning protocol does not stop due to continuous rotation between views, the computer device reduces the number of image frames to be captured at each energy threshold, for example, only one frame at each energy threshold, to ensure that the energy bin information corresponding to each energy threshold is obtained in a shorter time.

In the above embodiments, the trigger signal is the signal that the computer device periodically sends to the photon counting detector based on multiple sets of energy thresholds. Of course, in some embodiments, the trigger signal may also be sent by the photon counting detector or triggered by the computer device and the photon counting detector together. In some embodiments, the trigger signal is a periodic signal determined by the photon counting detector based on multiple sets of energy thresholds and an internal clock. In some embodiments, the trigger signal may be a signal periodically sent by a computer device to the photon counting detector based on multiple sets of energy thresholds as well as a periodic signal determined by the photon counting detector based on multiple sets of energy thresholds and an internal clock. Of course, in some embodiments, the trigger signal may be triggered by other control components of the CT device.

The embodiments of the present disclosure first determine the total number of viewing angles for reconstruction of the photon counting detector, and then periodically send the trigger signal to the photon counting detector according to the total number of viewing angles for reconstruction and the number of groups of energy thresholds, so the method provided in the present embodiments may be used in the photon counting detector under the continuous scanning protocol, It further improves the time-domain matching between the energy bin information.

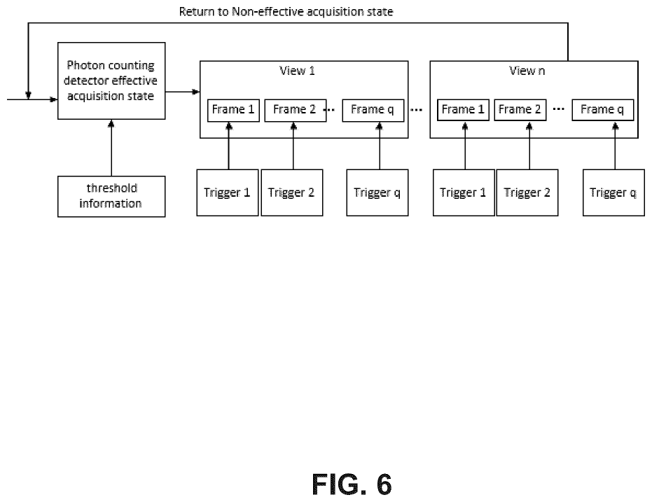

FIG. 6 shows the workflow diagram of a photon counting detector in an embodiment of the present disclosure under the continuous scanning protocol. As shown in FIG. 6, the photon counting detector may change from the non-effective acquisition state to the effective acquisition state after receiving the threshold information sent by the computer device. Then, the computer device periodically sends trigger signals to the photon counting detector based on the number of energy thresholds in each group of energy thresholds and the total number of viewing angles for reconstruction of the photon counting detector.

In FIG. 6, P represents the preset number of viewing angles in the continuous scan protocol, which is an integer greater than 0. k is the total number of energy thresholds in the threshold information, and q is the number of groups of energy thresholds.

For example, the photon counting detector A, with a preset viewing angle P=120, the total number of energy thresholds in the threshold information k=6, each group of energy thresholds corresponds to two energy thresholds, the number of energy threshold groups q=3, that is, the threshold information includes Threshold-1-Threshold-6, a total of three groups of energy thresholds, taking a single scan of rotation 360° as an example. Then the angular step of the collected image is 1°(based on 360° /120/3). When the CT device is rotated 0°, the computer device sends trigger signal one to the photon counting detector A, which then adjusts the threshold voltages of the threshold comparator one and threshold comparator two respectively according to the voltages corresponding to threshold one and threshold two, It then captures one frame of image at threshold voltage one and threshold voltage two respectively. When the CT device is turned to 1°, the computer device sends trigger signal two to the photon counting detector A, at which point the photon counting detector A adjusts the threshold voltages of the threshold comparator one and threshold comparator two respectively according to the voltages corresponding to threshold three and threshold four. Repeat the previous set of acquisition timing to capture one frame of image at threshold voltage three and threshold voltage four respectively. When the CT device turned to 2°, the computer device sent trigger signal three to the photon counting detector A, which adjusted the threshold voltages of the threshold comparator one and threshold comparator two respectively according to the voltages corresponding to threshold five and threshold six. Repeat the previous set of acquisition timing to capture one frame of image at threshold voltage five and threshold voltage six respectively. At this point, one view (i.e., view one) image capture is completed. It should be noted that since the CT device is in a continuous rotation state, the rotation angle of the CT device corresponding to view one is the angle corresponding to image frames 1-3 [0°,3°. It should be noted that image frames 1-3 actually include six frames of images, that is, each threshold voltage corresponds to one frame of image, and each frame of image contains one energy bin information. As mentioned above, the photon count detector A acquires the energy bin information corresponding to Threshold-1-Threshold-6 respectively in view one. Then, with the CT device rotating 3° and entering view two, the computer device will continue to send trigger signals to the photon counting detector A until the CT device rotates 6°, when the photon counting detector A acquires the energy bin information corresponding to Threshold-1-Threshold-6 of view two. The rotation angle range of the CT device corresponding to view two is [3°, 6°), and at this view two, image frames 1-3 are also captured. So the photon counting detector A may obtain P*k (i.e., P*q*2) frames at P=120 views and return to the non-valid capture state after obtaining P*k frames at P=120 views.

In some embodiments of the present disclosure, the sampling frequency comprises a first sampling frequency, the first sampling frequency is configured to indicate a time interval between each step angle of the photon counting detector. The trigger signal includes a first trigger signal. The said sending the trigger signal, periodically, to the photon counting detector based on the number of groups of the energy thresholds and the sampling frequency corresponding to the stepwise scanning protocol, comprises: determining, according to the first sampling frequency, whether the photon counting detector has entered the field of view. Upon determining that the photon counting detector has entered the said field of view, sending the first trigger signal directly to the photon counting detector. Wherein the first trigger signal is configured to trigger the photon counting detector within an acquisition cycle of one field of view, sequentially, to adjust a threshold voltage of at least one threshold comparator disposed in the photon counting detector to a voltage corresponding to each of the multiple sets of energy thresholds.

In some embodiments of the present disclosure, the sampling frequency comprises a second sampling frequency. The second sampling frequency is configured to indicate a data acquisition frequency of the photon counting detector at each of the said step angle. The said to adjust a threshold voltage of at least one threshold comparator disposed in the photon counting detector to a voltage corresponding to each of the multiple sets of energy thresholds, comprises: to adjust a threshold voltage of at least one threshold comparator disposed in the photon counting detector to a voltage corresponding to each of the multiple sets of energy thresholds based on the second sampling frequency.

The following will describe the execution process of the scanning method for CT device in a photon counting detector.

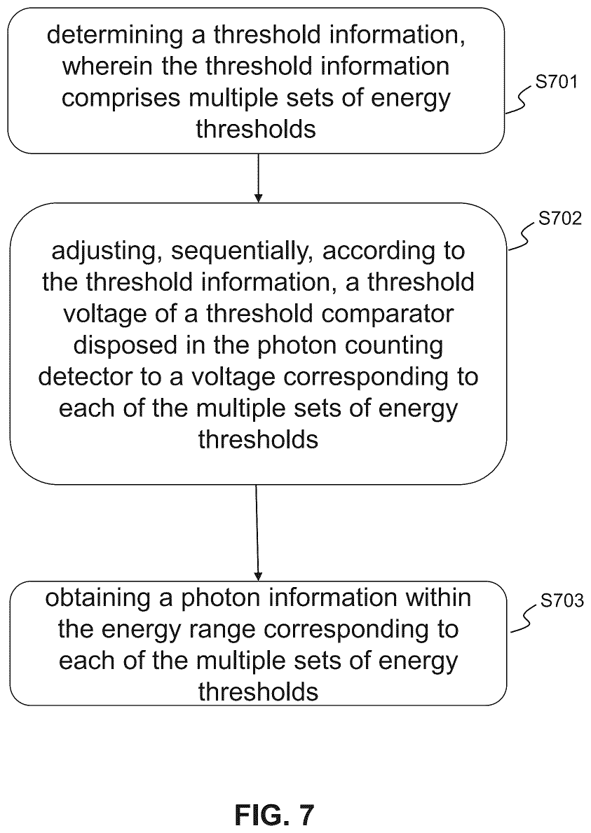

FIG. 7 is a schematic diagram of the flow of another scanning method for CT device in an embodiment of the present disclosure, which may be applied to the photon counting detector shown in FIG. 1. In one embodiment, as shown in FIG. 7, the following steps $701 to S703 are included.

Step S701, determining a threshold information, wherein the threshold information comprises multiple sets of energy thresholds.

In this embodiment, the photon counting detector needs to determine the threshold information before obtaining the energy bin information corresponding to multiple energy thresholds, that is, the photon information of the energy range corresponding to multiple energy thresholds. The threshold information may be either the information sent by the computer device to the photon counting detector or the information stored in advance by the user to the photon counting detector.

The threshold information includes multiple sets of energy thresholds, which are used to instruct the photon counting detector to obtain the energy bin information corresponding to each set of energy thresholds. For an introduction to threshold information, refer to the above step S201, which will not be elaborated here.

Step S702, adjusting, sequentially, according to the threshold information, a threshold voltage of a threshold comparator disposed in the photon counting detector to a voltage corresponding to each of the multiple sets of energy thresholds.

In this embodiment, after the photon counting detector has determined the threshold information, it may adjust the threshold voltages of its own threshold comparators in sequence to the voltages corresponding to each energy threshold based on the threshold information.

In some embodiment cases, after determining the threshold information, the photon counting detector may, based on its internal clock control, adjust the threshold voltages of its threshold comparator 1 and threshold comparator 2 to the voltages corresponding to Threshold-1 and threshold-2 respectively after entering each viewing angle. Then adjust the threshold voltages of its own threshold comparator 1 and threshold comparator 2 to Threshold-3 and Threshold-4 respectively. Finally, adjust the threshold voltages of your own threshold comparator 1 and threshold comparator 2 to the voltages corresponding to threshold 5 and threshold 6 respectively, and so on.

In some embodiments, the photon counting detector may also receive the trigger signal sent by the computer device, thereby using the trigger signal to successively adjust the threshold voltage of the threshold comparator within itself to the voltage corresponding to each group of energy thresholds. For example, after receiving the trigger signal one sent by the computer device, the photon counting detector adjusts the threshold voltages of its own threshold comparator 1 and threshold comparator 2 to the voltages corresponding to Threshold-1 and Threshold-2 respectively. Then adjust the threshold voltages of its own threshold comparator 1 and threshold comparator 2 to Threshold-3 and Threshold-4 respectively, and so on, to obtain the energy bin information corresponding to different energy thresholds.

Among them, the trigger signal may be based on level trigger, edge trigger, or pulse trigger. And the trigger signal may be a signal determined by a computer device outside the photon counting detector, or by the photon counting detector itself through an internal clock, or by other software-hardware combinations.

The process by which the photon counting detector adjusts the threshold voltage of the threshold comparator in sequence to the voltage corresponding to each energy threshold may be referred to the steps in the above embodiments and will not be repeated here.

It is understandable that if the trigger signal is the one determined by the photon counting detector according to its own scanning protocol and internal clock, the photon counting detector should at least include a processing unit, which includes registers. Among them, the processing pnit may be a central processing unit (CPU), and it may also include a digital signal processing (DSP), field-programmable gate array (FPGA), or other programmable logic devices.

S703, obtaining a photon information within the energy range corresponding to each of the multiple sets of energy thresholds.

In this embodiment, in conjunction with FIG. 3, after the photon counting detector successively adjusts the threshold voltage of its own threshold comparator to the voltage corresponding to each energy threshold, the photon counting detector may obtain the energy bin information corresponding to each energy threshold, that is, the photon information corresponding to the energy range corresponding to each energy threshold.

In one embodiment, the photon counting detector comprises a mapping relationship between the energy threshold of the photon counting detector stored on the photon counting detector and the voltage of the threshold comparator. The said adjusting, sequentially, according to the threshold information, a threshold voltages of a threshold comparator disposed in the photon counting detector to a voltages corresponding to each of the multiple sets of energy thresholds, comprises: converting, according to the multiple sets of energy thresholds, and the mapping relationship between the multiple sets of energy thresholds and the threshold voltages of the threshold comparator, the threshold voltages of the threshold comparator to a voltages of the threshold comparator corresponding to each of the multiple sets of energy thresholds.

The scanning method for CT device provided in the present embodiment first determines the threshold information, which includes multiple sets of energy thresholds, and then, based on the threshold information, adjusts the threshold voltages of the threshold comparators in the photon counting detector in sequence to the voltages corresponding to each set of energy thresholds, thereby obtaining the photon information of the energy range corresponding to each set of energy thresholds. Since the photon counting detector may adjust the threshold voltage of the threshold comparator, it may obtain the voltages corresponding to each energy threshold in a single scan of the photon counting detector, thereby obtaining the energy bin information corresponding to each energy threshold, that is, the energy range photon information corresponding to each energy threshold. Since the energy range photon information of multiple energy thresholds is obtained within a single scan time, the scanning method for CT device provided in this implementation may improve the time-domain matching of the energy range photon information corresponding to multiple energy thresholds, thereby improving the quality of image reconstruction.

It should be understood that although the steps in the flowcharts involved in the embodiments described above are shown in sequence as indicated by the arrows, these steps are not necessarily performed in sequence as indicated by the arrows. Unless explicitly stated herein, there are no strict sequence restrictions on the execution of these steps, and they may be executed in other sequences. Moreover, at least some of the steps involved in the flowcharts of the embodiments described above may include multiple steps or stages, which are not necessarily completed at the same time but may be executed at different times, and the order of execution of these steps or stages is not necessarily sequential. Instead, they may be executed alternately or in turn with other steps or at least part of steps or stages within other steps.

Based on the same inventive concept, the embodiments of the present disclosure also provide a photon counting detector for implementing the scanning method for CT device mentioned above and a computer device. The implementation of the solution provided by the photon counting detector and the computer device is similar to the implementation described in the above method, so the specific limitations in one or more of the photon counting detector and computer device embodiments provided below may be found in the above text for the limitations of the scanning method for CT device, and will not be elaborated here.

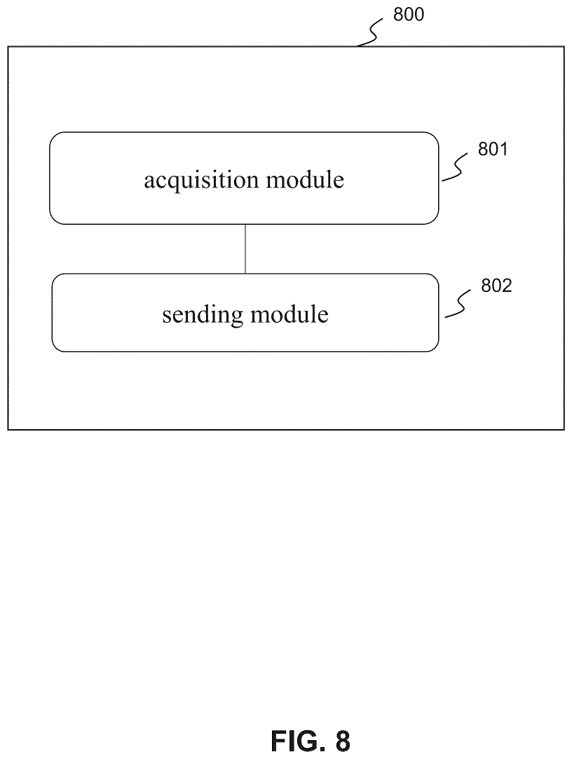

FIG. 8 shows a block diagram of the structure of a computer device in an embodiment of the present disclosure, as shown in FIG. 8, a computer device 800 in an embodiment of the present disclosure comprises: the acquisition module 801 and the transmission module 802.

The acquisition module 801 is configured to obtain the configured threshold information, wherein the threshold information includes multiple sets of energy thresholds.

The sending module 802 is configured to send trigger signals to the photon counting detector based on multiple sets of energy thresholds, where the trigger signals are used to trigger the photon counting detector to adjust the threshold voltages of the threshold comparators in the photon counting detector in sequence to the voltages corresponding to each set of energy thresholds.

The computer device provided in this embodiment first acquires the configured threshold information, which includes multiple sets of energy thresholds, and then sends trigger signals to the photon counting detector based on the multiple sets of energy thresholds. The trigger signal is configured to trigger the photon counting detector to adjust the threshold voltage of the threshold comparator in the photon counting detector to the voltage corresponding to each energy threshold in sequence. In a single scan of the current photon counting detector, the threshold voltage of the threshold comparator does not change. That is to say, assuming the photon counting detector may obtain energy bin information corresponding to up to two energy thresholds in a single scan, if the user wants energy bin information corresponding to four energy thresholds, the photon counting detector has to perform two separate single scans. The first scan acquires the energy bin information corresponding to energy threshold 1 and energy threshold 2, and the second scan acquires the energy bin information corresponding to energy threshold 3 and energy threshold 4. Since there is at least one single scan time between the first and second scans. As a result, the time-domain matching between the energy bin information corresponding to multiple energy thresholds in the current scanning device is poor.

In the embodiment of the present disclosure, by sending a trigger signal to the photon counting detector and using the trigger signal to trigger the photon counting detector to adjust the threshold voltage of the threshold comparator, the voltages corresponding to each group of energy thresholds may be obtained in a single scan of the photon counting detector, thereby obtaining the energy bin information corresponding to each energy threshold. Therefore, the computer device provided in the present implementation may improve the time-domain matching of energy bin information corresponding to multiple energy thresholds, thereby improving the quality of image reconstruction.

In some embodiments, the transmitting module 802 is configured to periodically send trigger signals to the photon counting detector based on multiple sets of energy thresholds and the scanning protocol of the photon counting detector.

In some embodiments, the transmission module 802 includes a first transmitting unit.

The first transmitting unit is configured to periodically send the trigger signal to the photon counting detector according to the number of groups of energy thresholds and the sampling frequency and/or sampling period corresponding to the stepwise scanning protocol, if the scanning protocol is a stepwise scanning protocol.

In one embodiment, the sampling frequency includes the first sampling frequency and/or the second sampling frequency. The first sampling frequency is configured to indicate the time interval between each step angle of the photon counting detector. The second sampling frequency is configured to indicate the data acquisition frequency of the photon counting detector at each step angle.

In one embodiment, the sampling period includes the time intervals between each step angle of the photon counting detector.

In one embodiment, the transmitting module 802 also includes a second transmitting unit.

The second transmitting unit is configured to periodically send a trigger signal to the photon counting detector based on the number of groups of energy thresholds if the scanning protocol is a continuous scanning protocol.

In one embodiment, the second transmitting unit comprises: the determination sub-unit and the sending sub-unit.

The determination sub-unit is configured to determine the total number of viewing angles for reconstruction of the photon counting detector.

Send the sub-unit, which is configured to periodically send the trigger signal to the photon counting detector based on the total number of viewing angles for reconstruction and the number of groups of viewing angles for reconstruction.

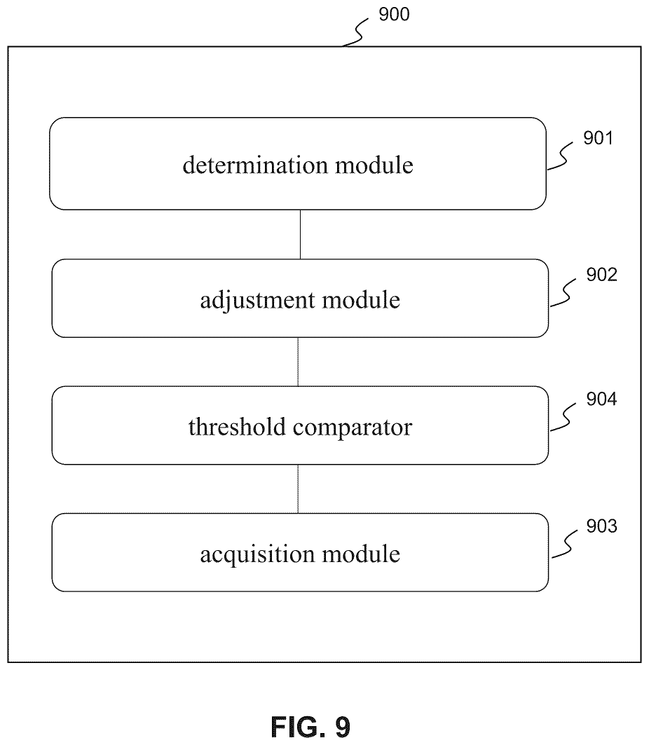

FIG. 9 shows a block diagram of the structure of a photon counting detector in an embodiment of the present disclosure, in which a photon counting detector 900 is provided, including: determination module 901, adjustment module 902, acquisition module 903 and threshold comparator 904. Threshold comparator 904 is any existing threshold comparator, which is not limited in the present disclosure.

The determination module 901, is configured to determine a threshold information, wherein the threshold information includes multiple sets of energy thresholds.

The adjustment module 902, connected to the determination module and the threshold comparator respectively, is configured to adjust a threshold voltage of the threshold comparator disposed in the photon counting detector to a voltage corresponding to each group of the energy thresholds in sequence according to the threshold information.

The acquisition module 903, connected to the threshold comparator, is configured to obtain the photon information of the energy range corresponding to each group of the energy thresholds.

For the photon counting detector provided in the present embodiment for the CT device, the determination module 901 first determines the threshold information, which includes multiple sets of energy thresholds, and then adjusts module 902 to adjust the threshold voltages of the threshold comparators in the photon counting detector in sequence to the voltages corresponding to each set of energy thresholds based on the threshold information. The module 903 acquires the photon information of the energy range corresponding to each energy threshold based on the voltage of the threshold comparator 904. Since the photon counting detector may adjust the threshold voltage of the threshold comparator, the photon counting detector may obtain the voltages corresponding to each energy threshold in a single scan of the photon counting detector, thereby obtaining the energy bin information corresponding to each energy threshold, that is, the photon information of the energy range corresponding to each energy threshold. Since the energy range photon information of multiple energy thresholds is obtained within the time of a single scan, the scanning device for CT device provided in the present implementation may improve the time-domain matching of the energy range photon information corresponding to multiple energy thresholds, thereby improving the quality of image reconstruction.

In some embodiments, the threshold information also includes the voltage of the threshold comparator corresponding to the energy threshold. The photon counting detector may also include a memory that stores the mapping relationship between the energy threshold of the photon counting detector and the voltage of the threshold comparator. Threshold information includes multiple sets of energy thresholds, each of which may be mapped to the voltage of the corresponding threshold comparator. In some embodiments, the mapping relationship between the multiple sets of energy thresholds stored by the photon counting detector and the voltage of the threshold comparator may be utilized to convert the received multiple sets of energy thresholds into the voltage of the corresponding threshold comparator. Of course, the voltage of the threshold comparator corresponds to the threshold voltage of the threshold comparator. By using different threshold voltages, the photon information of the energy range corresponding to each energy threshold may be obtained to complete the image acquisition of multiple groups of different energy ranges.

In one embodiment, the adjustment module is also configured to adjust, periodically, the threshold voltages of the threshold comparator disposed in the photon counting detector, sequentially, to the voltages corresponding to each of the multiple sets of energy thresholds, according to the multiple sets of energy thresholds and a scanning protocol of the photon counting detector.

In one embodiment, an energy spectrum CT system is provided, which comprises a computer device as described in any of the above embodiments and/or a photon counting detector as described in any of the above embodiments.

In one embodiment, the spectral CT system also includes a frame. The frame is rotatably set in the energy spectrum CT system. Specifically, the frame includes a rotor, and the photon counting detector is fixedly set on the rotor of the frame, and the photon counting detector is set to rotate with the rotor. The position of the object being scanned is fixed during the image scan. In one embodiment, the rack may also be fixedly set in an energy spectrum CT system, with the photon counting detector fixedly set on the frame, and the photon counting detector fixedly set during the image scan, with the scanned object set to be rotatable.

In one embodiment, the computer device in the energy spectrum CT system acquires threshold information configured including multiple sets of energy thresholds, and then sends a trigger signal to the photon counting detector in the energy spectrum CT system according to the scanning protocol of the multiple sets of energy thresholds and the photon counting detector.

In one embodiment, if the scanning protocol is a stepwise scanning protocol, the computer device periodically sends the trigger signal to the photon counting detector according to the number of groups of energy thresholds and the sampling frequency and/or sampling period corresponding to the stepwise scanning protocol. according to the determination that the scanning protocol is a stepwise scanning protocol, send the trigger signal, periodically, to the photon counting detector based on the number of groups of the energy thresholds and the sampling frequency corresponding to the stepwise scanning protocol. According to the determination that the scanning protocol is a continuous scanning protocol, determine a total number of viewing angles for reconstruction of the photon counting detector, then, send the trigger signal, periodically, to the photon counting detector according to the total number of viewing angles for reconstruction and the number of groups of the energy threshold.

Further, the photon counting detector in the energy spectrum CT system uses the trigger signal to adjust the threshold voltage of the threshold comparator in itself to the voltage corresponding to each energy threshold in sequence, thereby obtaining the energy bin information corresponding to each energy threshold.

Among them, computer device may be personal computers, laptops, smart phones, tablet computers and portable wearable devices set outside the photon counting detector, or CPU, DSP, FPGA or other programmable logic devices set inside the photon counting detector, or some may be set outside the photon counting detector. Some are placed inside the photon counting detector, and the outside and inside of the photon counting detector may communicate with each other.

In some embodiments, the energy spectrum CT system comprises a tube for emitting X-rays, wherein an energy-bin information corresponding to each energy threshold is defined for an energy range starting at the energy threshold and ending at an energy corresponding to a voltage of the tube.

In some embodiments, the spectral CT system also includes a reconstruction device for image reconstruction based on the bin information of each energy.

In some embodiments, the spectral CT system also includes a storage device for storing data during the scanning process and the reconstruction process.

In some embodiments, the spectral CT system also includes a display device for displaying the reconstructed image.

The individual modules in the photon-counting detector and computer equipment mentioned above may be implemented in whole or in part through software, hardware, or a combination thereof. These modules may be embedded in hardware form within or independently of the processor in the computer device, or stored in software form in the memory of the computer device so that the processor may invoke and perform the operations corresponding to these modules.

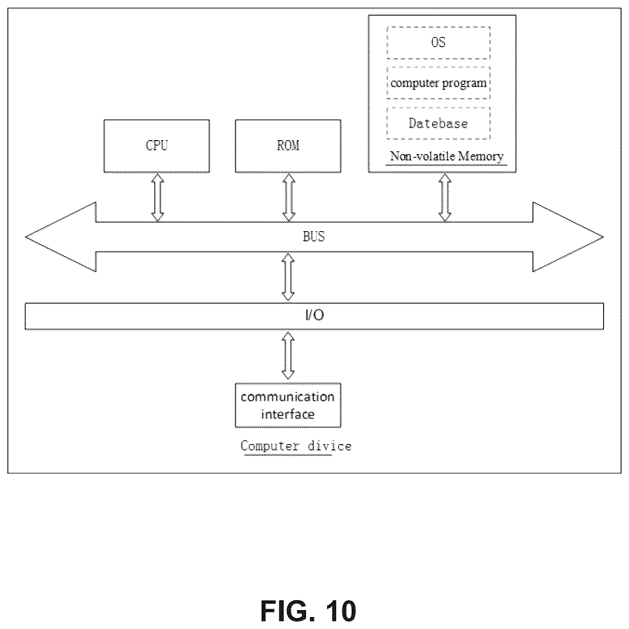

FIG. 10 is a schematic diagram of the internal structure of a computer device in an embodiment of the present disclosure, in which a computer device, which may be a server, is provided, and its internal structure diagram may be shown in FIG. 10. The computer device comprises a processor, a memory and a network interface connected via a system bus. Among them, the processor of the computer device is configured to provide computing and control capabilities. The memory of the computer device includes non-volatile storage media and internal memory. The non-volatile storage medium stores the operating system, computer programs and databases. The internal memory provides the environment for the operation of the operating system and computer programs in the non-volatile storage medium. The database of the computer device is configured to store relevant data. The network interface of the computer device is configured to communicate with external terminals via network connection. The computer program is executed by the processor to implement a scanning method for CT devices.

It may be understood by those skilled in the art that the structure shown in FIG. 10 is merely a block diagram of a part of the structure related to the present disclosure and does not constitute a limitation to the computer equipment to which the present disclosure is applied, which may include more or fewer components than shown in the figure, or combine certain components, or have different component arrangements.

In one embodiment, a computer device is provided, including a memory and a processor, in which a computer program is stored, and the processor performs the following steps when executing the computer program: obtaining configured threshold information, wherein the threshold information comprises multiple sets of energy thresholds. Sending, according to the multiple sets of energy thresholds, a trigger signal to the photon counting detector, wherein the trigger signal is configured to trigger the photon counting detector, sequentially, to adjust a threshold voltage of at least one threshold comparator disposed in the photon counting detector to a voltage corresponding to each of the multiple sets of energy thresholds.

In one embodiment, the processor also performs the following steps when executing the computer program: sending the trigger signal, periodically, to the photon counting detector according to the multiple sets of energy thresholds and a scanning protocol of the photon counting detector.

In one embodiment, the processor also performs the following steps when running the computer program: according to the determination that the scanning protocol is a stepwise scanning protocol, sending the trigger signal, periodically, to the photon counting detector based on the number of groups of the energy thresholds and the sampling frequency corresponding to the stepwise scanning protocol.

In one embodiment, the processor also performs the following steps when running the computer program: according to the determination that the scanning protocol is a continuous scanning protocol, sending the trigger signal, periodically, to the photon counting detector according to the number of groups of the energy threshold.

In one embodiment, the following steps are also implemented when the processor runs the computer program: determining a total number of viewing angles for reconstruction of the photon counting detector; sending the trigger signal, periodically, to the photon counting detector according to the total number of viewing angles for reconstruction and the number of groups of the energy threshold.

In one embodiment, the processor also implements the following steps while running the computer program: determining a threshold information, wherein the threshold information comprises multiple sets of energy thresholds; adjusting, sequentially, according to the threshold information, a threshold voltage of a threshold comparator disposed in the photon counting detector to a voltage corresponding to each of the multiple sets of energy thresholds; obtaining a photon information within the energy range corresponding to each of the multiple sets of energy thresholds.

In one embodiment, a non-volatile computer-readable storage medium is provided on which an executable instruction is stored, which, when executed by the processor, enables the processor to perform the following steps: obtaining configured threshold information, wherein the threshold information comprises multiple sets of energy thresholds; sending, according to the multiple sets of energy thresholds, a trigger signal to the photon counting detector, wherein the trigger signal is configured to trigger the photon counting detector, sequentially, to adjust a threshold voltage of at least one threshold comparator disposed in the photon counting detector to a voltage corresponding to each of the multiple sets of energy thresholds.

In one embodiment, when the executable instruction is executed by the processor, the processor further performs the following steps: periodically sending the trigger signal to the photon counting detector according to the multiple sets of energy thresholds and the scanning protocol of the photon counting detector.

In one embodiment, when the executable instruction is executed by the processor, the processor further performs the following steps: if the scanning protocol is a stepwise scanning protocol, a trigger signal is sent periodically to the photon counting detector according to the number of groups of the energy thresholds and the sampling frequency and/or sampling period corresponding to the stepwise scanning protocol.

In one embodiment, when the executable instruction is executed by the processor, the processor further performs the following steps: if the scanning protocol is a continuous scanning protocol, a trigger signal is sent periodically to the photon counting detector according to the number of groups of the energy thresholds.

In one embodiment, when the executable instruction is executed by the processor, the processor also performs the following steps: determine the total number of viewpoints for reconstruction of the photon-counting detector; trigger signals are periodically sent to the photon counting detector according to the total number of viewing angles for reconstruction and the number of groups of the energy threshold.

In one embodiment, when the executable instruction is executed by the processor, the processor also performs the following steps: determine the threshold information, wherein the threshold information includes multiple sets of energy thresholds; according to the threshold information, the threshold voltage of the threshold comparator in the photon counting detector is successively adjusted to the voltage corresponding to the energy thresholds of each group; obtaining the photon information of the energy range corresponding to the energy thresholds of each group.

In one embodiment, a computer program product is provided, including a computer program which, when executed by a processor, implements the following steps: obtaining configured threshold information, wherein the threshold information comprises multiple sets of energy thresholds; sending, according to the multiple sets of energy thresholds, a trigger signal to the photon counting detector, wherein the trigger signal is configured to trigger the photon counting detector, sequentially, to adjust a threshold voltage of at least one threshold comparator disposed in the photon counting detector to a voltage corresponding to each of the multiple sets of energy thresholds.

It may be understood by those skilled in the art that the implementation of all or part of the processes in the above-mentioned embodiments may be accomplished by instructing the relevant hardware through a computer program. The computer program may be stored in a non-volatile computer-readable storage medium, and when executed, it may include the processes of the embodiments of the above-mentioned methods. Any reference to memory, database or other medium used in the embodiments provided in the present disclosure may include at least one of non-volatile and volatile memory. Non-volatile Memory may include Read-Only memory (ROM), magnetic tape, floppy disk, flash Memory, optical memory, high-density embedded non-volatile memory, ReRAM, Magnetoresistive Random Access Memory (MRAM), Ferroelectric Random Access Memory (FRAM), Phase Change Memory (PCM), graphene memory, etc. Volatile Memory may include Random Access Memory (RAM) or external cache memory, etc. As an illustration rather than a limitation, RAM may take various forms, such as Static Random Access Memory (SRAM) or Dynamic Random Access Memory (DRAM), etc. The databases involved in the embodiments provided in the present disclosure may include at least one of a relational database and a non-relational database. Non-relational databases may include blockchain-based distributed databases, etc., but not limited to these. The processors involved in the embodiments provided in the present disclosure may be general-purpose processors, central processing units, graphics processing units, digital signal processing units, programmable logic units, quantum computing-based data processing logic units, etc., not limited to these.

The technical features of the above embodiments may be combined in any way. For the sake of conciseness, not all possible combinations of the technical features of the above embodiments are described. However, as long as there is no contradiction in the combination of these technical features, they should be considered within the scope of this specification.

The embodiments described above only express several embodiments of the present disclosure, which are described more specifically and in detail, but should not be construed as limiting the scope of the present disclosure. It should be noted that for those skilled in the art, several variations and improvements may be made without departing from the conception of the present disclosure, all of which fall within the scope of protection of the present disclosure. Therefore, the scope of protection of the present disclosure shall be based on the appended claims.

What is claimed is:

1. A scanning method for a CT device, which comprises a photon counting detector, wherein the method comprises:
obtaining configured threshold information, wherein the threshold information comprises multiple sets of energy thresholds;
sending, according to the multiple sets of energy thresholds, a trigger signal to the photon counting detector, wherein the trigger signal is configured to trigger the photon counting detector, sequentially, to adjust a threshold voltage of at least one threshold comparator disposed in the photon counting detector to a voltage corresponding to each of the multiple sets of energy thresholds.

2. The method according to claim 1, wherein the sending, according to the multiple sets of energy thresholds, a trigger signal to the photon counting detector, comprises:
sending the trigger signal, periodically, to the photon counting detector according to the multiple sets of energy thresholds and a scanning protocol of the photon counting detector.

3. The method according to claim 2, wherein the sending the trigger signal, periodically, to the photon counting detector according to the multiple sets of energy thresholds and a scanning protocol of the photon counting detector, comprises:
according to the determination that the scanning protocol is a stepwise scanning protocol, sending the trigger signal, periodically, to the photon counting detector based on the number of groups of the energy thresholds and the sampling frequency corresponding to the stepwise scanning protocol.

4. The method according to claim 3, wherein the sampling frequency comprises at least one of a first sampling frequency and a second sampling frequency;
the first sampling frequency is configured to indicate a time interval between each step angle of the photon counting detector;
the second sampling frequency is configured to indicate a data acquisition frequency of the photon counting detector at each of the said step angle.

5. The method according to claim 4, wherein the first sampling frequency is a reciprocal of the time intervals between each step angle of the photon counting detector.

6. The method according to any one of claims 2 to 5, wherein the sending the trigger signal, periodically, to the photon counting detector according to the multiple sets of energy thresholds and a scanning protocol of the photon counting detector, comprises:
according to the determination that the scanning protocol is a continuous scanning protocol, sending the trigger signal, periodically, to the photon counting detector according to the number of groups of the energy threshold.

7. The method according to claim 6, wherein the sending a trigger signal, periodically, to the photon counting detector according to the number of groups of the energy threshold, comprises:
determining a total number of viewing angles for reconstruction of the photon counting detector;
sending the trigger signal, periodically, to the photon counting detector according to the total number of viewing angles for reconstruction and the number of groups of the energy threshold.

8. The method according to claim 3, wherein the sampling frequency comprises a first sampling frequency, the first sampling frequency is configured to indicate a time interval between each step angle of the photon counting detector;
the trigger signal includes a first trigger signal;
sending the trigger signal, periodically, to the photon counting detector based on the number of groups of the energy thresholds and the sampling frequency corresponding to the stepwise scanning protocol, comprises:
determining, according to the first sampling frequency, whether the photon counting detector has entered the field of view;
upon determining that the photon counting detector has entered the said field of view, sending the first trigger signal directly to the photon counting detector, wherein the first trigger signal is configured to trigger the photon counting detector within an acquisition cycle of one field of view, sequentially, to adjust a threshold voltage of at least one threshold comparator disposed in the photon counting detector to a voltage corresponding to each of the multiple sets of energy thresholds.

9. The method according to claim 8, wherein the sampling frequency comprises a second sampling frequency, the second sampling frequency is configured to indicate a data acquisition frequency of the photon counting detector at each of the said step angle;
said to adjust a threshold voltage of at least one threshold comparator disposed in the photon counting detector to a voltage corresponding to each of the multiple sets of energy thresholds, comprises: to adjust a threshold voltage of at least one threshold comparator disposed in the photon counting detector to a voltage corresponding to each of the multiple sets of energy thresholds based on the second sampling frequency.

10. The method according to any one of claims 1-9, wherein the trigger signal carries the threshold information.

11. The method according to claim 3, wherein the sampling frequency comprises at least one of a first sampling frequency and a second sampling frequency;
wherein, the first sampling frequency characterizes a rotation angle per sampling of the CT device;
the second sampling frequency is configured to indicate a data acquisition frequency of the photon counting detector at each of the said step angle.

12. The method according to claim 6 or 7, wherein the sending the trigger signal, periodically, to the photon counting detector, comprises sending the trigger signal to the photon counting detector for each preset angle of rotation of the CT device, wherein, a step the preset angle is determined from a total rotation angle, a total number of viewing angles, and the number of groups of the energy threshold for each viewing angle.

13. A scanning method for a CT device, wherein the CT device comprises a photon counting detector, wherein the method comprises:
determining a threshold information, wherein the threshold information comprises multiple sets of energy thresholds;
adjusting, sequentially, according to the threshold information, a threshold voltage of a threshold comparator disposed in the photon counting detector to a voltage corresponding to each of the multiple sets of energy thresholds;
obtaining a photon information within the energy range corresponding to each of the multiple sets of energy thresholds.

14. The method according to claim 13, wherein the adjusting, sequentially, according to the threshold information, a threshold voltage of a threshold comparator disposed in the photon counting detector to a voltage corresponding to each of the multiple sets of energy thresholds, comprises:
periodically, adjusting the threshold voltages of the threshold comparator disposed in the photon counting detector, sequentially, to the voltages corresponding to each of the multiple sets of energy thresholds, according to the multiple sets of energy thresholds and a scanning protocol of the photon counting detector.

15. The method according to claim 13 or 14, wherein,
adjusting, sequentially, according to the threshold information, a threshold voltages of a threshold comparator disposed in the photon counting detector to a voltages corresponding to each of the multiple sets of energy thresholds, comprises: converting, according to the multiple sets of energy thresholds, and the mapping relationship between the multiple sets of energy thresholds and the threshold voltages of the threshold comparator, the threshold voltages of the threshold comparator to a voltages of the threshold comparator corresponding to each of the multiple sets of energy thresholds.

16. A computer device, comprising: a memory and a processor, wherein the memory has a computer program stored thereon, wherein the processor performs the steps of the method described in any one of claims 1 to 12 while executing the computer program.

17. A non-volatile computer-readable storage medium, having an executable instruction stored thereon, wherein the executable instruction, when executed by a processor, implements the method described in any one of claims 1 to 12.

18. A photon counting detector, comprising:
a threshold comparator;
a determination module, is configured to determine a threshold information, wherein the threshold information includes multiple sets of energy thresholds;
an adjustment module, connected to the determination module and the threshold comparator respectively, is configured to adjust a threshold voltage of the threshold comparator disposed in the photon counting detector to a voltage corresponding to each group of the energy thresholds in sequence according to the threshold information;
an acquisition module, connected to the threshold comparator, is configured to obtain the photon information of the energy range corresponding to each group of the energy thresholds.

19. The photon counting detector according to claim 18, wherein the adjustment module is also configured to adjust, periodically, the threshold voltages of the threshold comparator disposed in the photon counting detector, sequentially, to the voltages corresponding to each of the multiple sets of energy thresholds, according to the multiple sets of energy thresholds and a scanning protocol of the photon counting detector.

20. An energy spectrum CT system, comprising:
a photon counting detector according to claim 18 or 19; and/or
a computer device according to claim 16.
